(19) United States
(12) Patent Application Publication
(10) Pub. No.: US 2006/0292661 A1
(43) Pub. Date: Dec. 28, 2006

(54) Electrochemical sensing assays involving drug metabolizing enzymes

(75) Inventor: Richard Gilbert, Essex (GB)

Correspondence Address: HELLER EHRMAN WHITE & MCAULIFFE LLP, 1717 RHODE ISLAND AVE, NW, WASHINGTON, DC, 20036-3001, US

(73) Assignee: E2V Technologies (UK) Limited, Essex (GB)

(21) Appl. No.: 10/554,786

(22) PCT Filed: Apr. 30, 2004
(86) PCT No.: PCT/GB04/01806

§ 371 Date: Mar. 10, 2006

(30) Foreign Application Priority Data
Apr. 30, 2003 (GB) 0309905.8

(51) Int. Cl. C12Q 1/30; C07F 17/02

(52) U.S. Cl. 435/27; 205/777.5; 546/2; 544/228; 549/3; 556/144

(57) ABSTRACT

Use of an electrochemical mediator to transfer electrons from an electrode to molecules of a mammalian oxidative drug metabolizing enzyme (DME) in solution is described, in particular to carry out assays to determine metabolism of a candidate drug by the DME. Transfer of electrons by the mediator is carried out in the absence of a reductase enzyme for the DME molecules. The mediator is in solution with the DME molecules and/or immobilized to the electrode. Where the mediator is immobilized to the electrode, this may form a protective layer on the electrode thereby reducing or preventing denaturation of the DME molecules by direct contact with the electrode. Electrodes and electrochemical reaction chambers for use in the assays are also described.

This invention relates to use of electrochemical mediators, in particular for determining metabolism of candidate drugs by oxidative drug-metabolising enzymes (DMEs), and to electrodes, electrochemical reaction chambers, and devices for use in such assays.

A key area of interest in the pharmaceutical industry is the prediction of how drugs are metabolised in the body. One of the main drug metabolism processes, phase I oxidative metabolism, is primarily mediated by either the cytochrome P450 (Cyp) or flavin monooxygenase (FMO) families of enzymes, the so-called oxidative drug-metabolising enzymes (DMEs). The primary physiological role of the DMEs is to add a hydroxyl moiety to foreign molecules, thus facilitating their metabolic degradation. The catalytic reaction can be summarised as:
RH+O₂+2H⁺+2e⁻→ROH+H₂O

where RH can be one of a large range of possible substrates.

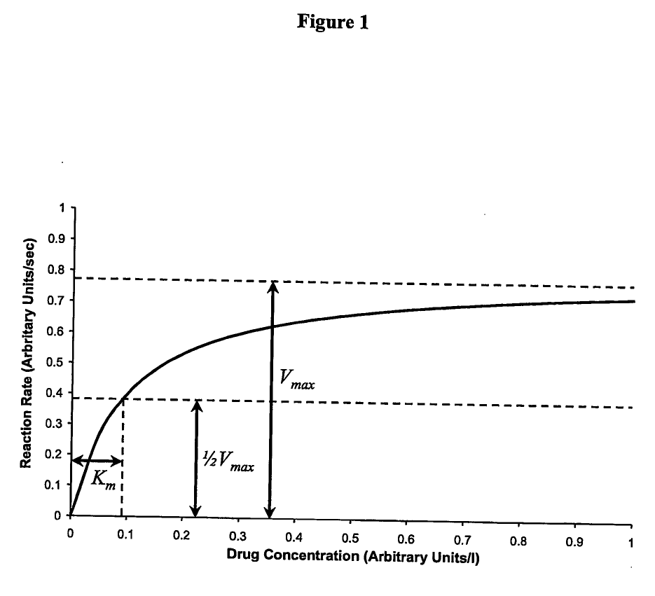

When investigating metabolism of candidate drugs by a DMB, the key parameters to measure are the maximal rate of this reaction (Vₘₐₓ) for different candidate drugs, and the concentration of candidate drug which gives half of Vₘₐₓ, a parameter known as Kₘ (FIG. 1). Vₘₐₓ is a measure of how fast the DME can process the candidate drug, and Kₘ indicates the binding affinity between the candidate drug and the DME. The ratio of these two parameters (Vₘₐₓ/Kₘ) is termed the intrinsic clearance CLᵢₙₜ, which may be thought of as the clearance rate of candidate drug from liver plasma devoid of the influence of blood flow or binding. Good candidate drugs will have a very low CLᵢₙₜ, meaning they are resistant to degradation by DMEs; bad candidate drugs, with a high CLᵢₙₜ, would be degraded very rapidly.

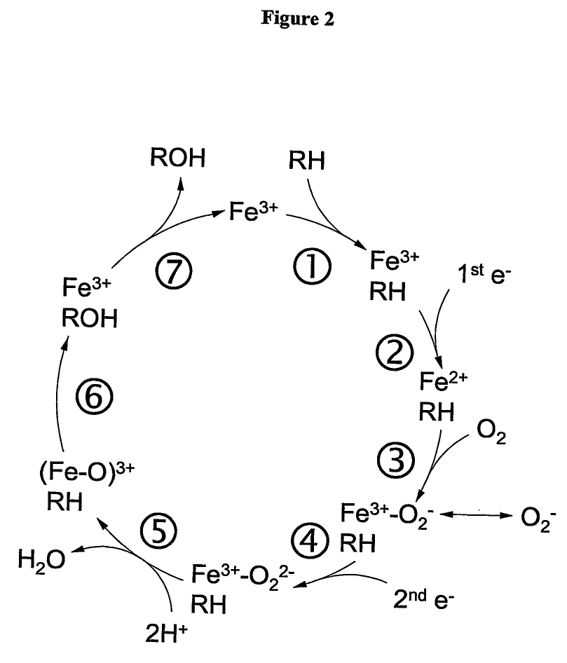

The reactions catalysed by redox enzymes such as DMEs are driven by the transfer of electrons. The generally accepted Cyp catalytic cycle is shown in FIG. 2. The reaction begins when the substrate binds to the active site (1). If the reaction is to proceed further, the substrate must displace a water molecule that is normally co-ordinated to the haem iron atom in unbound Cyp. This is accompanied by a change in the spin of the Fe³⁺ ion from a low spin (½) state in which the five 3d electrons are maximally paired, to a high spin ( 5/2) state in which the electrons are maximally unpaired. This in turn causes a change in the redox potential of the iron of approximately 100 mV, which is sufficient to make the reduction of the iron by the redox-partner of the Cyp (usually NADPH or NADH) thermodynamically favourable (2). The reduction step is followed by the binding of an O₂ molecule to a separate site adjacent to the Fe³⁺ ion (3). This state is not stable, and is easily autooxidised releasing O₂⁻. If, however, the transfer of a second electron occurs (4), the catalytic reaction continues. The O₂²⁻ reacts with protons from the surrounding solvent to form H₂O (which is released), leaving an activated oxygen atom (5). This may then react with the substrate molecule (6) resulting in a hydroxylated form of the substrate (7) which is then released from the active site.

The electrons which drive this reaction cycle are normally supplied in vivo by redox partners with the aid of appropriate oxidoreductase enzymes. In the case of the DMEs, the redox partner is usually nicotinamide adenosine dinucleotide phosphate (NADPH), which switches between oxidised (NADP+) and reduced states. Current in vitro DME assays require a reasonably complex reaction mixture which is able to regenerate the redox partners in the appropriate oxidation state. For example, recombinant P450s have been used in combination with recombinant P450 reductase and NADPH. While NADPH consumption can be followed spectrophotometrically, the coupling between electron flow and product formation is variable. Thus, NADPH consumption may not be a reliable indicator of metabolism of a candidate drug. A further disadvantage of such assays is that P450 reductase is expensive.

It has been shown that it is possible to drive redox enzyme reactions artificially by supplying the electrons directly using electrodes in an electrochemical reaction chamber.

For example, systems have been developed in which fusion proteins comprising an NADPH-cytochrome P450 reductase and a cytochrome P450 are driven electrochemically from a platinum electrode by means of a mediator, cobalt sepulchrate (Estabrook, R. W. et al., Endocrine Research 22(4), 665-671 (1996)). However, disadvantages of such systems are that the cytochrome P450 must be provided as a fusion protein with a cytochrome P450 reductase, and reliable results may not be obtained because they rely on coupling between the reductase and the cytochrome P450.

WO 00/22158 describes attachment of a cytochrome P450 to a graphite electrode so that electrons can be supplied to the enzyme without the need for a P450 reductase. This document also discloses use of a modified gold electrode to supply electrons to a cytochrome P450 in solution.

Despite these developments, however, DMEs have not yet been fully exploited electrochemically mainly due to the difficulty in achieving an efficient transfer of electrons from the electrode to the enzymes' active sites. A further problem is that proteins readily denature on electrode surfaces, particularly if the electrode is metallic. Most protein surfaces carry a large number of functional groups which are able to bind to metallic surfaces with a fairly high affinity, and any protein molecules touching such a surface are likely to adhere to it and unfold, thereby becoming inactive and ‘poisoning’ the electrode with an insulating layer.

International patent application no. PCT/GB03/02756 is a co-pending application filed between the priority and filing date of the present application. It is directed to use of electrochemical sensing for predicting drug metabolism by a DME. It is stated that one way to maximise the transfer of electrons from the electrode(s) to the catalytic site within the enzyme is to immobilise the enzyme at the surface of the electrode. In one embodiment the electrode is covalently modified. The surface of an electrode of, for example, metal (typically though not exclusively gold) or graphite, is modified by the covalent addition of chemical groups to make it more amenable for the transfer of electrons to proteins. One technique involves the use of organothiloate compounds (containing an SH group) in conjunction with a gold electrode. The thiol group forms a strong bond to the metal surface, with the rest of the molecule providing suitable functional groups for interacting with the protein.

In a further embodiment of PCT/GB03/02756 microporous electrolyte membranes are used. These are mechanically and chemically stable polymer gels with high ionic conductivity, coating the surface of an electrode in the form of a thin layer. The polymers comprising the gel should be chosen to provide a suitable environment for trapping the proteins within their matrix, such as a high proportion of carboxylic acid groups (for proteins with many positively-charged surface residues), amine groups (for proteins with many negative charges at the surface), or aliphatic groups (for proteins with largely hydrophobic surfaces).

A further embodiment of PCT/GB03/02756 involves use of lipid membranes. Natural CYP enzymes are usually found attached to biological membranes, since they almost exclusively contain a region which acts as an anchor within a phospholipid bilayer. Indeed, the CYPs used in analytical laboratories are generally modified to remove this anchor domain, thus allowing the enzyme to be solubilised. The affinity of CYPs for lipid bilayer membranes provide a means of anchoring them at the surface of an electrode. Suitable membranes may be constructed using long-chain fatty acids, lipids, or similar molecules, deposited on the surface.

The present invention relates to the transfer of electrons from the electrode(s) to the catalytic site of the DME when this is in solution and not immobilised to the electrode(s).

According to the invention there is provided use of an electrochemical mediator to transfer electrons from an electrode to molecules of an oxidative drug metabolising enzyme (DME) in solution, wherein the mediator is in solution.

According to the invention transfer of electrons by the mediator from the electrode to the DME molecules is carried out in the absence of a reductase enzyme for the DME molecules.

The DME molecules are large molecules compared to mediator molecules, and so diffuse only slowly onto and away from the electrode surface. Because the mediator molecules are able to diffuse much more quickly than the DME molecules, they can shuttle between the electrode surface and the DME molecules in bulk solution, carrying one or more electrons. The rate of mediated electron transfer is, therefore, much greater than could be achieved by the DME molecules themselves. The addressable volume of the electrode is thereby increased and hence the signal to noise ratio is dramatically improved compared to that with the DME molecules alone.

The term “oxidative DME” is used herein to include Cyps and FMOs. Other, as yet unidentified oxidative DMEs may also be found, for example as a result of the human genome project, and are also included within the scope of this term. The oxidative DME may be a naturally occurring oxidative DME, or a recombinant oxidative DME, or a derivative thereof that retains drug metabolising activity. Examples of such derivatives are enzymes that do not include amino acid residues required for membrane binding so that their solubility is increased. The oxidative DME should be a class II drug metabolising enzyme (in vivo these enzymes require a flavoprotein NADPH-dependent reductase, and include mammalian and some bacterial P450s), rather than a class I drug metabolising enzyme (in vivo these enzymes require an NADPH-dependent reductase and an iron-sulphur protein, and include most bacterial P450s and the mitochondrial steroid-metabolising enzymes). Preferably, the oxidative DME is a human oxidative DME. In some circumstances, however, it may be desired to use a non human oxidative DME, such as a rat or a mouse oxidative DME.

The term “mediator” includes any species having a reversible redox couple with an E^{o} value close to that of the DME.

The electrode may be any suitable electrically conductive material, preferably graphite or metal, and most preferably gold.

According to a preferred aspect of the invention, the electrode is an unmodified electrode, preferably an unmodified metal electrode, particularly an unmodified gold electrode.

According to an alternative preferred aspect of the invention there is provided use of an electrochemical mediator to transfer electrons from an electrode to molecules of an oxidative drug metabolising enzyme (DME) in solution, wherein the mediator comprises a first mediator in solution, and a second mediator immobilised to the electrode optionally by means of a linker.

In embodiments of this alternative preferred aspect, molecules of a first mediator are immobilised to the electrode, and molecules of a second mediator are in bulk solution. In these embodiments, the enzyme molecules may obtain the electrons either from the mediator molecules in bulk solution or directly from the molecules immobilised to the electrode.

The second mediator may be immobilised to the electrode by any suitable means. The second mediator may be covalently or non-covalently immobilised to the electrode. Where a linker is used, the linker may be covalently or non-covalently bound to the electrode, and the second mediator may be covalently or non-covalently bound to the inker.

Preferably the second mediator is non-covalently immobilised to the electrode or linker, and the first and second mediators have the same chemical structure. This can be achieved where a mediator comprising one or more functional groups capable of binding to the electrode or linker is provided in excess. In such embodiments there is sufficient mediator to bind to the electrode and to remain in solution with the DME molecules.

In other embodiments the first and second mediators may be different chemical compounds.

In other preferred embodiments the first mediator comprises a functional group that is capable of reacting with the electrode or linker to form a covalent bond with the electrode or linker, and the second mediator is the product of covalent binding of a chemical with the same structure as the first mediator to the electrode or linker.

Advantageously, the second mediator and/or the linker forms a protective layer on the electrode thereby reducing or preventing denaturation of the DMB molecules caused by direct contact of the DME molecules with the electrode. With such embodiments it is particularly preferred that the first and second mediators have the same chemical structure, or that the second mediator is the product of covalent binding of a chemical with the same structure as the first mediator to the electrode or linker. According to such embodiments a single type of mediator performs two functions: it provides an efficient means to carry electrons from the electrode to the DME molecules, and protects the DME molecules from denaturing on the surface of the electrode.

Where the second mediator and/or the linker forms a protective layer on the electrode, the density of the layer should be established such that molecules of the DME are sterically hindered from contacting the surface of the electrode. The protective layer should be formed over as much as possible of the surface of the electrode that contacts the solution in which the DME molecules are present. In practice, it is expected that it will be possible to coat up to approximately 80% of the surface area of the electrode that contacts the solution. The presence of impurities on the surface of the electrode are likely to prevent formation of a protective coating over 100% of the surface area.

In other preferred embodiments of the invention the electrode is coated with a substance through which molecules of the mediator, but not the DME molecules, can diffuse. Such embodiments have the advantage that denaturation of the DME molecules caused by direct contact with the electrode is reduced or prevented, but the mediator molecules can efficiently transfer electrons from the electrode to the DME molecules in solution.

In further preferred embodiments of the invention the electrode is coated with a substance through which the DME molecules cannot diffuse, and the second mediator is bound to the substance or trapped within it, thereby immobilising the second mediator to the electrode. It will be appreciated that the second mediator may be provided with a suitable functional group(s) so that the second mediator binds to the substance. Such embodiments may be particularly advantageous if they allow a relatively high concentration of second mediator molecules to be formed around the electrode since this should allow efficient transfer of electrons from the electrode to the DME molecules. According to these preferred embodiments the first and second mediators may have different chemical structures. However, preferably the first and second mediators have the same chemical structure, or the second mediator is the product of covalent binding of a chemical with the same structure as the first mediator to the substance.

Suitable coating substances include protein size exclusion gels, preferably polysaccharide gels such as Sephadex. Optionally the gel may be modified to increase its affinity for the electrode and/or the mediator. For example, the gel may be modified by conjugation to sulphur or pyridine groups to increase its affinity for gold electrodes.

For efficient formation of a protective coating, such as a monolayer, the mediator or substance preferably comprises one or more functional groups able to form a bond on the electrode's surface which is strong enough to immobilise the mediator molecules or substance at the operating voltage of the electrode. It will be appreciated that the functional group(s) will depend on the electrode material. Preferred metal binding groups for binding to metal electrodes include amides, amines, carboxylic acids, and heterocyclic groups such as thiophenes, or nitrogen containing heterocyclic groups such as pyridines, purines, or pyrimidines. For a gold electrode, suitable functional groups include (but are not limited to) thiols, thioethers, thiophenes, pyridine, nitrogen-containing heterocycles, carboxylic acids and most negatively-charged moieties.

Where the second mediator is immobilised to the electrode by means of a linker or substance that has low electrical conductivity (i.e. an insulator, such as a lipid), the distance between the electrode and mediator closest to the electrode should be no longer than 20 Angstroms, and preferably 15-18 Angstroms (for example a C₆ lipid). It is believed that distances greater than this will cause the rate at which electrons are able to pass from the electrode to the mediator to become limiting.

The mediator may be used in an electrochemical assay for determining whether a candidate drug, suitably a xenobiotic, is metabolised by the DME. If the candidate drug acts as a substrate for the DME, then turnover of the candidate drug by the DME will consume electrons (for example, a Cyp enzyme is expected to consume two electrons per candidate drug molecule if the reaction proceeds via the Cyp catalytic cycle shown in FIG. 2). The rate of consumption of electrons by the DMEE can be measured using an electrochemical reaction chamber provided the mediator transfers electrons from an electrode of the electrochemical reaction chamber to the DME at a rate which is at least as fast as the rate at which they are consumed by the DME (otherwise accurate measurement of the rate of consumption of electrons is not possible since the rate limiting step becomes the transfer of electrons). Ohm's law predicts that if increasing voltage is applied to the electrochemical reaction chamber a constant linear rise in current will occur if there is constant resistance. However, if the candidate drug acts as a substrate for the DME, a deviation from a constant linear rise in current will be seen as electrons are consumed by the reaction. This deviation can be used to calculate the rate of consumption of electrons by the DME and, therefore, the rate of turnover of the candidate drug by the DME. If this assay is performed for different concentrations of the candidate drug, Vmax and Km can be calculated.

A suitable assay comprises the following steps: i) providing an electrochemical reaction chamber comprising electrodes, a solution of DME molecules, an electrochemical mediator; and a candidate drug; ii) applying changing voltage to the electrochemical reaction chamber; iii) measuring current flowing through the electrochemical reaction chamber; and iv) determining from the measured current whether the candidate drug is metabolised by the DME.

The electrodes may be any suitable electrically conductive material, preferably graphite or metal, and most preferably gold. Preferably one or more reference electrodes are also included.

There is also provided according to the invention electrodes and electrochemical reaction chambers for use according to the invention.

In particular, there is provided according to the invention an electrode comprising an electrochemical mediator capable of transferring electrons from the electrode to molecules of a mammalian oxidative DME in solution, wherein the mediator is immobilised to the electrode, optionally by means of a linker, and the mediator and/or the linker forms a protective layer on the electrode thereby reducing or preventing denaturation of the DME molecules caused by direct contact of the DME molecules with the electrode.

There is also provided according to the invention an electrode comprising an electrochemical mediator capable of transferring electrons from the electrode to molecules of a mammalian oxidative DME in solution, wherein the electrode is coated with a substance through which the DME molecules cannot diffuse, and the mediator is bound to the substance or trapped within it, thereby immobilising the mediator to the electrode.

There is further provided according to the invention an electrochemical reaction chamber comprising at least two electrodes, an electrochemical mediator, and molecules of a DME in solution, wherein the mediator is in solution.

The mediator may comprise a first mediator in solution, and a second mediator immobilised to one or both of the electrodes optionally by means of a linker. The second mediator and/or the linker may form a protective layer on the electrode or electrodes thereby reducing or preventing denaturation of the DME molecules caused by direct contact of the DME molecules with the electrode or electrodes. Alternatively the electrode or electrodes may be coated with a substance through which the DME molecules cannot diffuse, and the second mediator is bound to the substance or trapped within it, thereby immobilising the second mediator to the electrode.

According to the invention there is also provided an electrochemical reaction chamber comprising at least two electrodes, an electrochemical mediator, and molecules of a DME in solution, wherein the mediator is in solution, and wherein one or both of the electrodes are coated with a substance through which molecules of the mediator, but not the DME molecules, can diffuse.

According to the invention there is also provided an electrochemical reaction chamber comprising at least two electrodes, an electrochemical mediator, and molecules of a DMR in solution, wherein the mediator is immobilised to one or both of the electrodes to form a protective layer thereby reducing or preventing denaturation of the DME molecules caused by direct contact of the DME molecules with the electrode or electrodes.

The invention also provides a device comprising a plurality of electrochemical reaction chambers of the invention, wherein each electrochemical reaction chamber comprises molecules of a different DME. Such devices may be used to determine the degree of processing of a candidate drug by each different DME to identify which DME(s) is likely to be primarily responsible for metabolism of the candidate drug.

The, or each electrochemical reaction chamber is preferably a micro-electrochemical reaction chamber.

According to a further aspect of the invention there is provided use of an electrochemical mediator to transfer electrons from an electrode to molecules of an oxidative drug metabolising enzyme (DM) in solution, wherein the mediator is immobilised to the electrode to form a protective layer thereby reducing or preventing denaturation of the DME molecules by direct contact with the electrode.

It will be appreciated that the mediator or mediators for use according to the invention should not act as a substrate or an inhibitor of the DME, otherwise accurate measurement of the rate of turnover of the candidate drug by the DME is not possible.

The mediator(s) must have a suitable redox potential for driving the enzyme-catalysed reactions. The importance of the redox potential of the mediator(s) and the operating voltage of the electrode of the electrochemical reaction chamber which supplies electrons to the mediator is explained below. Preferably, the working voltage of the electrode which supplies electrons in the electrochemical reaction chamber is more electronegative than the redox potential of the DME, and the redox potential of the mediator is less electronegative than the working voltage of the electrode, but more electronegative than the redox potential of the DME.

An oxidation-reduction (redox) reaction is one where one species loses electrons and another gains them. When a species gains electrons, it is being reduced. When a species loses electrons, it is being oxidized. In all redox reactions, reduction and oxidation occur together: one cannot happen without the other. The electrons flow from one species to the other: there is no net charge gain or loss.

The electrical force produced by an electrochemical cell is measured by the cell voltage, E. Cell voltage depends on the redox reactions occurring in the cell and the concentration of the reactants, but not on the number of electrons passing through the cell.

Since we can split a redox reaction into two parts, we can also define standard voltages for both the oxidation and reduction parts of the reaction, Eₐₓ⁰ and E_{red}⁰. We may arbitrarily pick the hydrogen reduction half reaction
2H⁺_{(aq)}+2e⁻→H_{2(g)}
to have E_{red}⁰=0, and measure all other half reaction voltages in relationship to it. Redox potentials are always given relative to such a reference reaction. In addition to the hydrogen ‘electrode’ shown above, a silver/silver chloride reference is also commonly used, and there are large tables published with the values of standard reduction voltages for half reactions with reference to standard electrode systems. The oxidation half reactions are simply the reaction run in reverse, and the half cell oxidation voltage is the negative of the reduction voltage. Note that the reference electrode in this sense is used to define a ‘baseline’ redox potential to enable redox differences to be quantified. Compounds whose redox potentials differ by, for example, 100 mV will show this same difference no matter what material is used for the reference electrode in the experimental electrochemical cell.

The standard voltage of a cell, E⁰, is the sum of the standard voltages of the oxidation and reduction half reactions. E⁰ is measured when all reactants are at 25° C. and at 1M concentration or 1 atm pressure. The use of the ‘0’ superscript indicates that the values are measured under standard conditions. The addition of an apostrophe, E⁰′; indicates that the values are measured under conditions standard for the system being studied. For biological systems, this would be at the relevant physiological conditions of pH, ionic concentration and temperature. To determine if a redox reaction is spontaneous, one should compute the voltage of the reaction. If the voltage is positive, the reaction is spontaneous, and if the voltage is negative, the reaction is not spontaneous.

For the general reaction
aA+bB<−>cC+dD
the equilibrium constant expression has the form
K=[C]^{c}[D]^{d}/[A]^{a}[B]^{b}
where K is the equilibrium constant for the reaction and [X] indicates the concentration of species X. The reaction quotient, Q, is expressed as
Q=[C]^{c}[D]^{d}/[A]^{a}[B]^{b}

The reaction quotient expression of a reaction has the same equation as the equilibrium constant expression for that reaction, however the reaction quotient is computed using the current concentrations, not the equilibrium ones, as indicated by the use of bold type. At equilibrium, Q=K. One use of Q is to determine which way a reaction will go, by computing Q using the current pressures or concentrations and comparing it to K for the reaction. If Q<K, then the reaction will move to the right, and if Q>K, then the reaction will move to the left.

Since the cell voltage E⁰ determines if the reaction in a cell is spontaneous or not, it clearly must be related to ΔG, the change in the Gibbs free energy. The relationship is
ΔG=−nFE
where n is the number of electrons that are exchanged during the balanced redox reaction and F is the Faraday constant, 9.648×10⁴ C/mol. At standard concentrations at 25° C., this equation can be written as
ΔG⁰=−nFE⁰

Redox reactions like all others can reach an equilibrium state. Since we have a relationship between E⁰ and ΔG⁰ as well as one between ΔG⁰ and K, we can derive a relationship between the cell voltage and the equilibrium constant. Since we have
ΔG⁰=−nFE⁰
and
   ΔG⁰=−RT.ln(K)
   we can combine the two equations into one:
   E⁰=(RT/nF).ln(K)

Under standard conditions, the term RT/F has the value of 0.0257 V, so we can simplify the above equation
E⁰=(0.0257/n).ln(K)

With the above equations, we can derive the value of the cell voltage from the equilibrium constant and vice versa.

We can combine the relationships between ΔG and E at non-equilibrium conditions to get a relationship between the two in much the same way that we can relate K and E at equilibrium. We have the relations
ΔG=ΔG⁰+RT.ln(Q)
ΔG=−nFE
ΔG⁰=−nFE⁰

Combining the three relations gives the Nernst equation
E=E⁰−(RT/nF).ln(Q)

This equation allows us to compute the cell voltage at any concentration of reactants and products and at any temperature. We can simplify the equation slightly by combining constants as before
E=E⁰−(0.0257/n).ln(Q)

In this invention, the mediator accepts electrons from the electrode, and is therefore being reduced. The degree to which this occurs can be calculated using the above equations, and it should be clear that the difference between the electrode voltage and the redox potential determines the relative proportions of oxidised and reduced mediator in the electrochemical reaction chamber. Thus, the redox potential of the mediator and the operating voltage of the electrode are of critical importance in driving the chemical reaction in the direction required. In a similar way, the mediator subsequently passes electrons to the DME molecules and is therefore being oxidised. Again, the difference between the redox potentials of the two molecules are crucial in determining the direction, and degree to which, the chemical reaction occurs.

A typical Cyp has a redox potential of −450 mV (vs. an Ag/AgCl reference electrode), so this value may be used to determine the preferred redox potentials for suitable mediators. According to the standard Cyp catalytic mechanism, the redox potential is lowered by a further 100 mV or so upon substrate binding. Each DME will have a characteristic redox potential, but it is likely that the preferred mediators will have potentials falling within the range +/−750 mV vs. an Ag/AgCl electrode.

The mediator participates in two electrochemical reactions:
Electrode⁻+Mediator<−>Electrode+Mediator⁻
Mediator⁻+DME<−>Mediator+DME⁻

Both of these reactions must move in the left-to-right direction at a rate that is faster than the rate of the reaction catalysed by the DME, summarised as
RH+O₂+2H⁺+2e⁻→H₂O+ROH

Where R represents the drug.

As has been described, the direction of the electrochemical reactions are determined by the changes in Gibbs free energy, which is related to chemical enthalpy and entropy by the following equation
ΔG=ΔH−T.ΔS

Where ΔH is the change in enthalpy, ΔS is the change in entropy, and T is the reaction temperature.

ΔH is primarily determined by interactions such as chemical (covalent) bonding, electrostatic interactions, hydrogen bonding and van der Waals interactions, not just between the two interacting molecules, but also between each interacting molecule and the solvent. Functional groups which would have a large impact on this component would therefore be those that produce strong interactions of the types listed previously. These include (but are not limited to) hydrogen bond donors and acceptors, such as hydroxyls, amines, amides, carboxylic acids, aromatic systems, heterocycles, enols, ethers, ketones, aldehydes, thiols, thioethers, plus halo-, nitro-, phospho- and sulphate groups, or thiol equivalents of these groups. Preferably the mediator comprises at least two or three of these groups.

ΔS is primarily determined by the degrees of freedom in the system, such as the total number of axes along which each molecule may move or rotate, the number of rotatable bonds, the degree of branching in chain-like groups, and the total number of atoms in the system. Again, this component needs to be considered not just between the two interacting molecules, but also between each interacting molecule and the solvent. Functional groups which would have a large impact on this component would therefore be those that contribute to the features listed previously. As before, these include (but are not limited to) amines, amides, carboxylic acids, aromatic systems, cyclic groups, particularly heterocycles, enols, ethers, ketones, aldehydes, thiols, thioethers, plus halo-, nitro-, phospho- and sulphate groups.

Many of the interactions described above contribute to the ‘hydrophobic interaction’ component of ΔG, which may be specifically influenced by functional groups such as aromatic systems, hydrogen-bond acceptors and/or donors, and charged groups.

It will be appreciated that the mediator should be capable of transferring electrons from the electrode to the DME at a rate which is at least as fast, preferably at least two times as fast, as the rate of consumption of electrons by the DME when a candidate drug is metabolised by the DME. If metabolism of the candidate drug is limited by the transfer of electrons, accurate measurement of the rate of turnover of the candidate drug by the DME is not possible since electron transfer to the DME then becomes the rate-limiting step.

Typically, a DME molecule will turnover approximately 10-100 substrate molecules per second. According to the Cyp catalytic mechanism two electrons are consumed for each molecule of substrate that is turned over. Thus, the mediator should be capable of transferring electrons from the electrode to the DME at a rate of at least 20 electrons per second, more preferably at least 40 electrons per second, most preferably at least 200 electrons per second.

Several characteristics influence the overall rate of transfer of electrons to the DME molecules. For embodiments of the invention in which the mediator is in solution it will be appreciated that the rate of diffusion of the mediator through the solution should be faster than the rate of diffusion of the DME through the solution. Other characteristics which are of equal or greater importance include the rate at which mediator and DME molecules collide in solution (determined primarily by their rates of diffusion and concentrations), the proportion of mediator molecules in the appropriate redox state (determined primarily by the working voltage of the electrode), the proportion of collisions which result in a transfer of electrons from a mediator to a DME molecule (determined primarily by their relative redox potentials and ΔG upon binding).

Many classes of organic molecule are suitable mediators for use according to the invention. These include (but are not limited to) metallocenes, flavins, quinones, and NADH, or redox active derivatives thereof.

Preferred mediators are metallocenes, in particular ferrocenes. Cobalt metallocenes and vanadium metallocenes are also preferred. Metallocenes have an unusual structure, in that a transition metal ion is sandwiched between two aromatic rings, such as the negatively charged cyclopentadienyl ion:

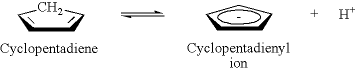

Two cyclopentadienyl rings can coordinate to an Fe²⁺ ion to form ferrocene, which may exist in either an oxidised or reduced state, thereby mirroring the characteristics iron in the active site of the DME haem groups:

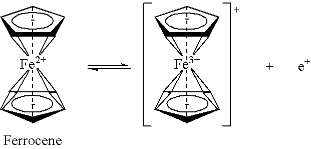

The ferrocenes in particular have appropriate redox potentials to be efficient mediators of charge for DMEs. They may carry substituent groups which can be used to optimise their binding characteristics to the enzymes and in addition they may be functionalised with appropriate chemical groups to allow them to bind tightly to the surface of the electrode, thereby forming the protective monolayer.

There are several positions on the ferrocene skeleton which may be functionalised by the addition of chemical groups in order to modulate the molecule's redox potential and other physico-chemical characteristics such as shape, size, hydrophobicity, charge, solubility and so on. These positions are indicated by the labels R₁ to R₁₀ in the Markush structure below.

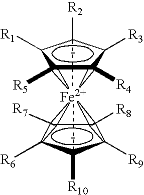

In ferrocene itself, all ten substituent positions are occupied by single hydrogen atoms. The substituent positions need not be independent. For example R₁ and R₂ might be joined together via a ring structure. The R positions are therefore simply indicators of where it is possible to vary the chemistry around the ferrocene core.

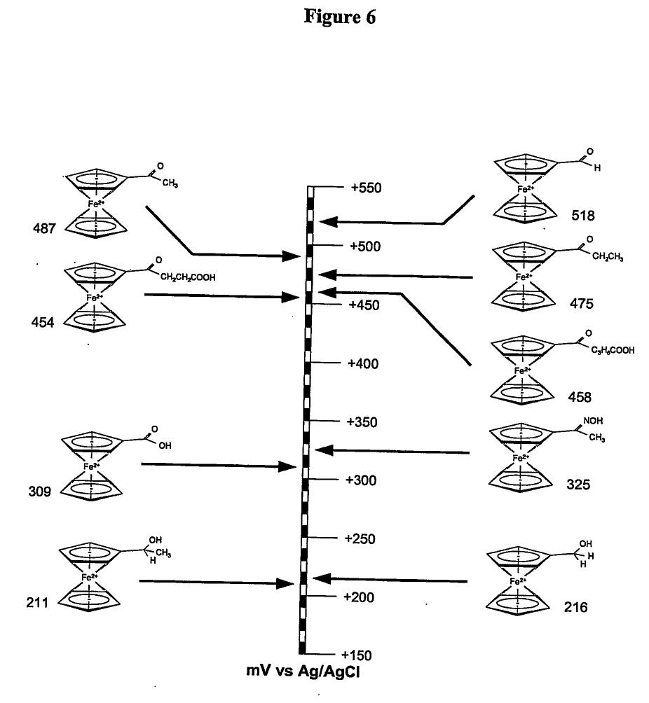

FIG. 6 shows a variety of mono-substituted ferrocene derivatives (i.e. where a single R group is not hydrogen) and their redox potentials with respect to a Ag/AgCl reference electrode. Electrons will transfer from a more electronegative molecule to a more electropositive one, with the difference in redox potentials determining the relative concentrations of the charged species at equilibrium. However, the molecules must not be too electronegative (or electropositive), since unfavourable electrostatic interactions may prevent the molecules physically interacting and hence may reduce the efficiency of electron transfer. There is therefore a requirement to optimise the redox potential of the mediator molecules in order to tailor them for each specific type of DME, which will themselves have characteristic and differing redox potentials. For example, the redox potential of the electron transport protein cytochrome C under physiological conditions is about +260 mV, whereas the redox potential of a typical Cyp is about −450 mV.

In addition to modulating the redox potential of the molecule, if at least one of the potential R groups carries a suitable functional group for binding to the electrode, then the mediator molecule will be able to form a protective monolayer on the electrode's surface. For example, it is well known that metallic gold has a particularly strong affinity for sulphur-containing groups such as thiols. If one of the R groups carries a thiol, it should therefore confer a strong gold-binding ability to the molecule. Provided this binding group is also able to support the ready transfer of electrons from the site of metal binding to the co-ordinated transition metal ion at the heart of the sandwich structure (e.g. by containing a delocalised electron system), then the monolayer should still have the ability to supply electrons to the fluid phase. There are many alternatives to a thiol group for binding the mediators to a gold electrode, and many alternatives to gold as the electrode material.

Preferred ferrocenes are compounds of the following formula: 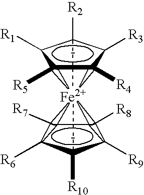
wherein R₁₋₁₀ are independently any of the following: hydrogen, an hydroxyl group, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, a halo-, nitro-, phospho-, or sulphate group, except where R₁₋₁₀ are all hydrogen.

Particularly preferred ferrocenes are compounds of the following formula: 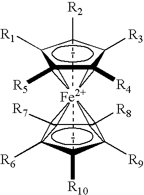
wherein R₁ is any of the following: an hydroxyl group, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, a halo-, nitro-, phospho-, or sulphate group, or a ferrocene; and R₂₋₁₀ are hydrogen.

For other preferred ferrocenes, R₃₋₁₀ are hydrogen, and R₁ and R₂ are independently any of the groups specified above for R₁.

As an example, thiomethylferrocene (with R₁=CH2SH and R₂ to R₁₀=H) is predicted to form a strong covalent bond on a gold surface. Predictions of the electrostatic potential at the molecular surface suggest that this molecule would also be able to abstract electrons from the metal and present them on its solvent-accessible face. Thus, thiomethylferrocene is predicted to have several characteristics which would make it eminently suitable to act as an electron-transport mediator for the DMEs.

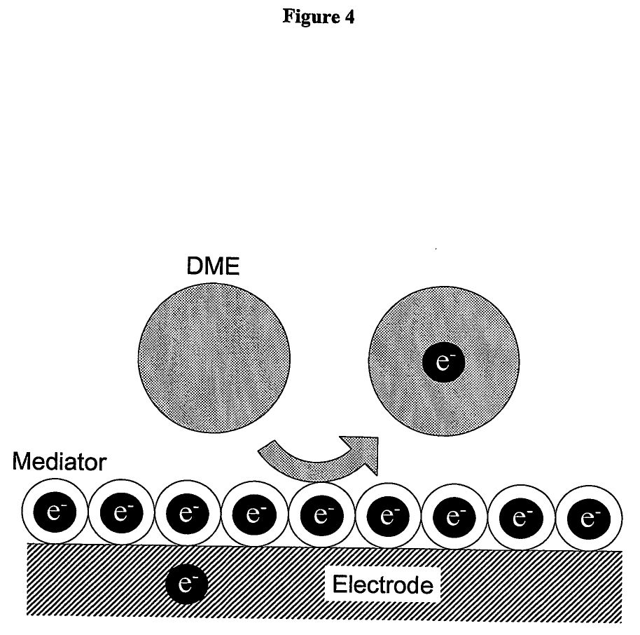

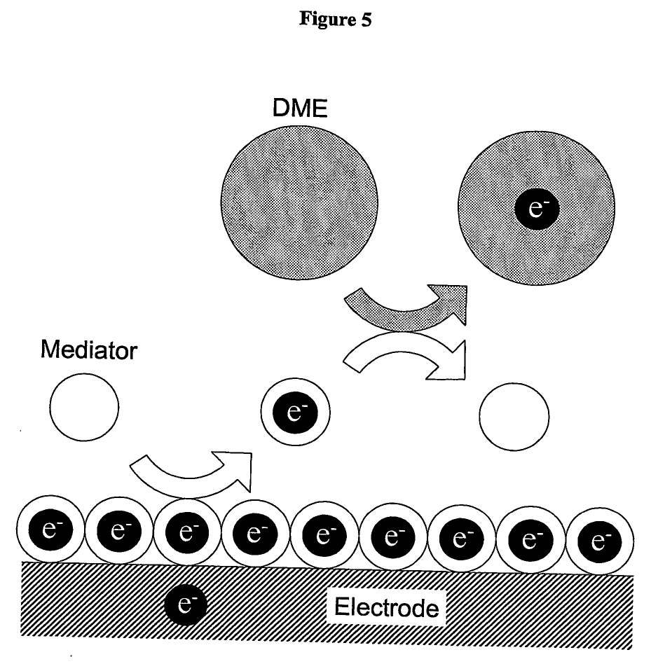

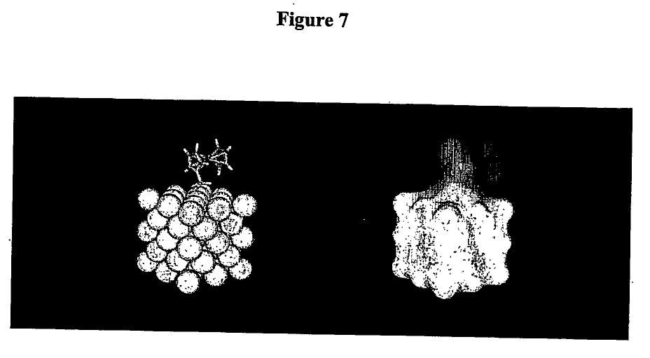

FIG. 7 shows a molecular model of thiomethylferrocene (represented as sticks) bonded to a small cluster of gold atoms (spheres) via the thiol group. To the right of the figure is a predicted solvent-accessible surface shaded according to electrostatic potential, with the darker shading being the most electronegative and the lighter shading being the least. It is evident that the thiomethylferrocene moiety is predicted to abstract electrons from the bulk metal and present them on its solvent-accessible surface, thereby providing a monolayer of spheres as represented in FIGS. 4 and 5.

Thiomethylferrocene is just an example of the type of molecule which may be suitable as a mediator for DME electrochemistry. In practice, each DME will require mediators with a characteristic set of redox and physico-chemical properties, and each type of electrode may require different binding groups to enable the protective monolayer to be formed.

Use of thiol containing groups for the mediator is preferred where the DME is a flavin monooxygenase, or an oxidative DME other than a-cytochrome P450.

Other preferred mediators are flavins, or redox active derivatives thereof. A generic structure for preferred flavin-based mediators is shown below: 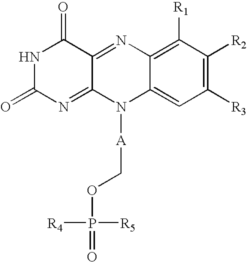
where R1, R2 and R3 are hydrogen, —CHO, —COCH₃, —COCH₂CH₃, —COC₃H₆COOH, —COCH₂CH₂COOH, —CNOHCH₃, —COOH, —CH₂OH, —CHOHCH₃, —OH, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, or a halo-, nitro-, phospho-, or sulphate group;

R4 and R5 are oxygen, hydroxyl, a nucleotide, a nucleoside, a thiol, a thioether, a thiophene, a pyridine, a nitrogen-containing heterocycle, a carboxylic acid, or a negatively-charged moiety; and

A is a spacer, suitably a derivatised or underivatised alkane, alkene, alkyne, ether, ester, amide, aromatic group, or heterocycle. An example is a ribityl sugar as found in the natural molecules flavin adenine dinucleotide (FAD) and flavin mononucleotide (FMN).

Preferred flavins are FAD and FMN, or redox active derivatives thereof.

A person skilled in the art will appreciate that candidate mediators for use according to the invention can be assayed using a DME and a known substrate of that DMF (for which the rate of reaction at a particular concentration of the substrate and DME, and/or the Km or Vmax values are known) in an electrochemical assay. If the candidate mediator is able to transfer electrons from the electrode to the DME at a rate which allows accurate calculation of the rate of reaction and/or the Km or Vmax value (this is judged by whether or not the calculated value is in agreement with the known value), then the candidate mediator can be used as a mediator according to the invention.

According to the invention there is also provided an assay for identifying an electrochemical mediator for use according to the invention which comprises:

i) providing an electrochemical reaction chamber comprising electrodes, a solution of DME molecules, a substrate for the DME molecules, and a candidate electrochemical mediator;

ii) applying changing voltage to the electrochemical reaction chamber;

iii) measuring current flowing through the electrochemical reaction chamber; and

iv) determining from the measured current the rate of reaction of the substrate with the DME; and

v) comparing the determined rate of reaction with a known rate of reaction of the substrate with the DME under corresponding conditions.

If the determined rate of reaction is in agreement with the known rate of reaction the candidate mediator is identified as a mediator for use according to the invention. The rate of reaction can be determined for different concentrations of the substrate so that Km and/or Vmax can be calculated and compared with the known value(s). Again, if the calculated Km or Vmax value is in agreement with the known value, the candidate mediator is identified as a mediator for use according to the invention.

In some embodiments of the invention, a mediator which is covalently immobilised to the electrode may be excluded.

In some embodiments of the invention, a mediator which is a microporous electrolyte membrane (a mechanically and stable polymer gel with high ionic conductivity) coating the surface of an electrode in the form of a thin layer may be excluded.

In some embodiments of the invention, a mediator which is a lipid membrane anchored at the surface of the electrode may be excluded.

Two of the many possible experimental approaches which are suitable for use in carrying out an assay of the invention are now described. The reactions are performed in an electrochemical reaction chamber. The DME and candidate drug is dissolved in aqueous solution (together with the mediators if these are in solution), preferably at a pH, temperature and ionic concentration which closely matches those of standard physiological conditions. Increasing voltage is applied to the electrochemical reaction chamber and the current is measured. The deviation in current from the constant linear rise in current predicted by Ohm's law if resistance is constant is used to calculate the reaction rate for different concentrations of candidate drug. The different reaction rates are then used to calculate the maximum rate (Vmax) of turnover of candidate drug by the DME, and the concentration of candidate drug (Km) which gives half of Vmax.

The electrochemical reaction chamber may be any suitable size. Bench scale vessels of a few millilitres volume are common, but our preferred reaction chamber would be incorporated into a microfluidics-scale device of a few tens or hundreds of nanoliters. The electrodes may be any suitable material, though our preference would be for gold.

Typical concentrations of the various components are likely to fall in the range 1-100 mM, though more dilute conditions would be preferable.

Linear Sweep Voltammetry (LSV)

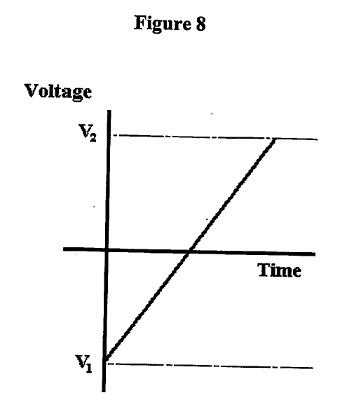

In linear sweep voltammetry the electrode voltage is scanned from a lower limit to an upper limit as shown in FIG. 8. The voltage scan rate (v) is calculated from the slope of the line. Clearly by changing the time taken to sweep the range the scan rate is altered.

The characteristics of the linear sweep voltammogram recorded depend on a number of factors including:

The rate of the electron transfer reaction(s)

The chemical reactivity of the electroactive species

The voltage scan rate

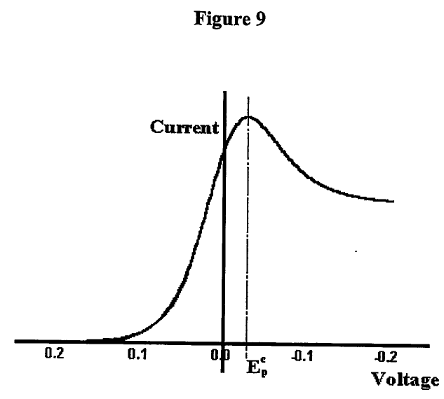

In LSV measurements the current response is plotted as a function of voltage rather than time, unlike potential step measurements. For example if we consider the Fe³⁺/Fe²⁺ system 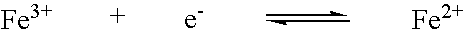
then the voltammogram shown in FIG. 9 would be seen for a single voltage scan using an electrolyte solution containing only Fe³⁺ resulting from a voltage sweep.

The scan begins from the left hand side of the current/voltage plot where no current flows. As the voltage is swept further to the right (to more reductive values) a current begins to flow and eventually reaches a peak before dropping. To rationalise this behaviour we need to consider the influence of voltage on the equilibrium established at the electrode surface. If we consider the electrochemical reduction of Fe³⁺ to Fe²⁺, the rate of electron transfer is fast in comparison to the voltage sweep rate. Therefore at the electrode surface an equilibrum is established identical to that predicted by thermodynamics. The Nernst equation

$$E = {E}^{θ} + \frac{RT}{nF} ln \frac{[ {Fe}^{3 +} ]}{[ {Fe}^{2 +} ]}$$

predicts the relationship between concentration and voltage (potential difference), where E is the applied potential difference and E⁰ is the standard electrode potential. So as the voltage is swept from V₁ to V₂ the equilibrium position shifts from no conversion at V₁ to full conversion at V₂ of the reactant at the electrode surface.

The exact form of the voltammogram can be rationalised by considering the voltage and mass transport effects. As the voltage is initially swept from V¹ the equilibrium at the surface begins to alter and the current begins to flow:

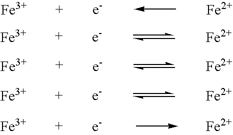

The current rises as the voltage is swept further from its initial value as the equilibrium position is shifted further to the right hand side, thus converting more reactant. The peak occurs, since at some point the diffusion layer has grown sufficiently above the electrode so that the flux of reactant to the electrode is not fast enough to satisfy that required by the Nernst equation. In this situation the current begins to drop just as it did in the potential step measurements.

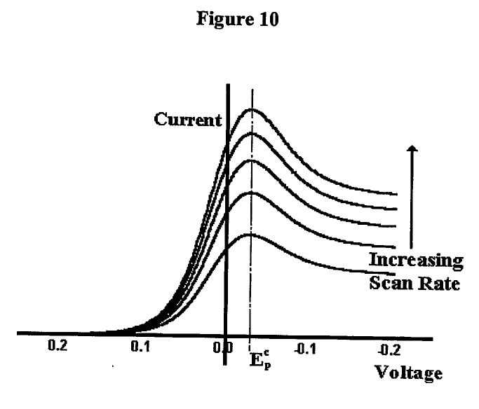

The above voltammogram was recorded at a single scan rate. If the scan rate is altered the current response also changes. FIG. 10 shows a series of linear sweep voltammograms recorded at different scan rates for an electrolyte solution containing only Fe³⁺. Each curve has the same form but it is apparent that the total current increases with increasing scan rate. This again can be rationalised by considering the size of the diffusion layer and the time taken to record the scan. Clearly the linear sweep voltammogram will take longer to record as the scan rate is decreased. Therefore the size of the diffusion layer above the electrode surface will be different depending upon the voltage scan rate used. In a slow voltage scan the diffusion layer win grow much further from the electrode in comparison to a fast scam Consequently the flux to the electrode surface is considerably smaller at slow scan rates than it is at faster rates. As the current is proportional to the flux towards the electrode the magnitude of the current will be lower at slow scan rates and higher at high rates. This highlights an important point when examining LSV (and cyclic voltammograms), although there is no time axis on the graph the voltage scan rate (and therefore the time taken to record the voltammogram) do strongly effect the behaviour seen. A final point to note from FIG. 10 is the position of the current maximum, it is clear that the peak occurs at the same voltage and this is a characteristic of electrode reactions which have rapid electron transfer kinetics. These rapid processes are often referred to as reversible electron transfer reactions.

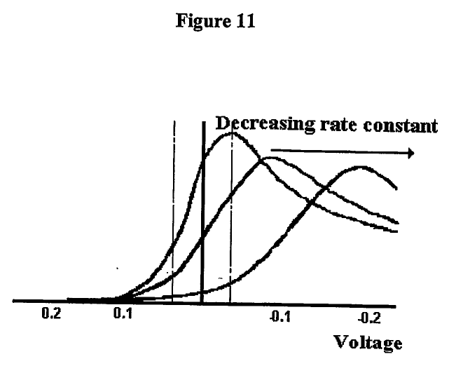

This leaves the question as to what would happen if the electron transfer processes were ‘slow’ (relative to the voltage scan rate). For these cases the reactions are referred to as quasi-reversible or irreversible electron transfer reactions. FIG. 11 shows a series of voltammograms recorded at a single voltage sweep rate for different values of the reduction rate constant (k_{red}).

In this situation the voltage applied will not result in the generation of the concentrations at the electrode surface predicted by the Nernst equation. This happens because the kinetics of the reaction are ‘slow’ and thus the equilibria are not established rapidly (in comparison to the voltage scan rate). In this situation the overall form of the voltammogram recorded is similar to that shown in FIG. 11, but unlike the reversible reaction now the position of the current maximum shifts depending upon the reduction rate constant (and also the voltage scan rate). This occurs because the current takes more time to respond to the applied voltage than the reversible case.

Cyclic Voltammetry

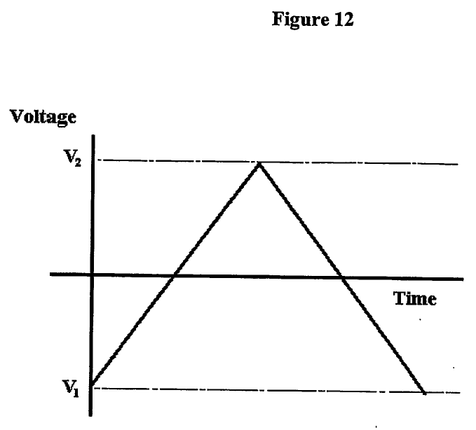

Cyclic voltammetry (CV) is very similar to LSV. In this case the voltage is swept between two values (see FIG. 12) at a fixed rate, however when the voltage reaches V₂ the scan is reversed and the voltage is swept back to V₁

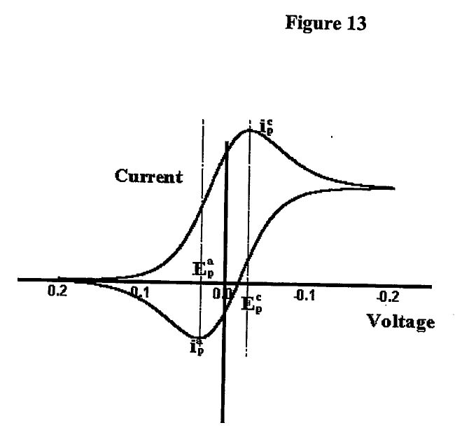

A typical cyclic voltammogram recorded for a reversible single electrode transfer reaction is shown in FIG. 13. Again the solution contains only a single electrochemical reactant. The forward sweep produces an identical response to that seen for the LSV experiment. When the scan is reversed we simply move back through the equilibrium positions gradually converting electrolysis product (Fe²⁺) back to reactant (Fe³⁺). The current flow is now from the solution species back to the electrode and so occurs in the opposite sense to the forward step but otherwise the behaviour can be explained in an identical manner. For a reversible electrochemical reaction the CV recorded has certain well defined characteristics:

I) The voltage separation between the current peaks is

$$Δ E = {E}_{p}^{a} - {E}_{p}^{c} = \frac{59}{n} mV$$

II) The positions of peak voltage do not alter as a function of voltage scan rate

III) The ratio of the peak currents is equal to one

$$\vert \frac{{i}_{p}^{a}}{{i}_{p}^{c}} \vert = 1$$

IV) The peak currents are proportional to the square root of the scan rate
iₚ^{a} and iₚ^{c}∝\sqrt{v}

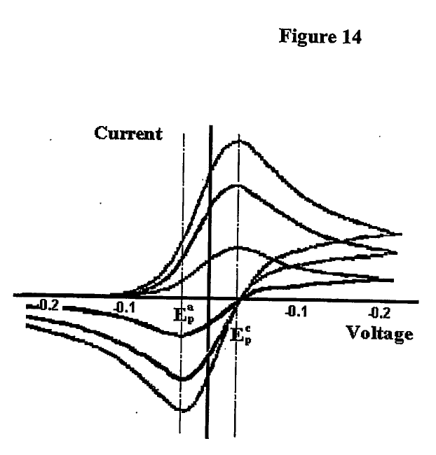

The influence of the voltage scan rate on the current for a reversible electron transfer can be seen in FIG. 14. As with LSV the influence of scan rate is explained for a reversible electron transfer reaction in terms of the diffusion layer thickness.

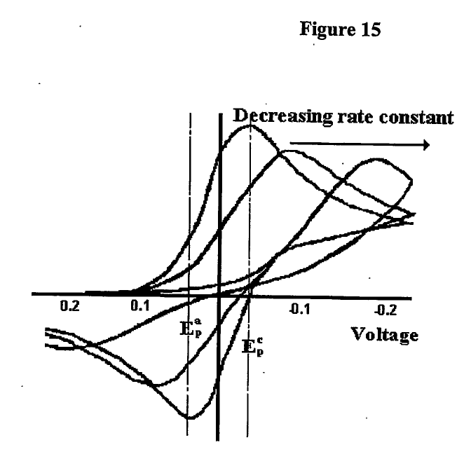

The CV for cases where the electron transfer is not reversible show considerably different behaviour from their reversible counterparts. FIG. 15 shows the voltammogram for a quasi-reversible reaction for different values of the reduction and oxidation rate constants. The first curve shows the case where both the oxidation and reduction rate constants are still fast, however, as the rate constants are lowered the curves shift to more reductive potentials. Again this may be rationalised in terms of the equilibrium at the surface is no longer establishing so rapidly. In these cases the peak separation is no longer fixed but varies as a function of the scan rate. Similarly the peak current no longer varies as a function of the square root of the scan rate. By analysing the variation of peak position as a function of scan rate it is possible to gain an estimate for the electron transfer rate constants.

Embodiments of the invention are now described, by way of example only, with reference to the accompanying drawings in which:

FIG. 1 shows the change in the rate of conversion of a drug by a DME as the concentration of the drug is increased;

FIG. 2 shows the generally accepted Cyp catalytic cycle;

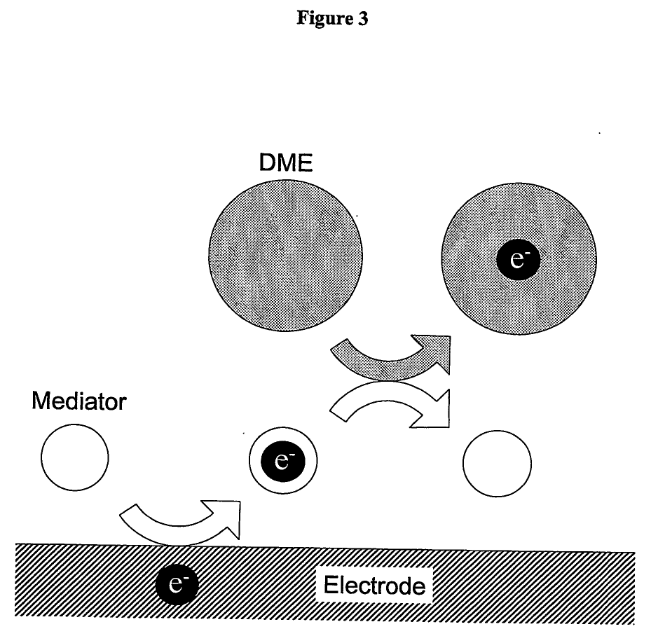

FIG. 3 shows an embodiment of the invention in which the mediator molecules are in solution;

FIG. 4 shows an embodiment of the invention in which the mediator molecules are immobilised to the electrode to form a protective layer on the surface of the electrode;

FIG. 5 shows an embodiment of the invention in which the mediator molecules are in solution and immobilised to the electrode to form a protective layer;

FIG. 6 shows a variety of mono-substituted ferrocene derivatives and their redox potentials with respect to an Ag/AgCl reference electrode;

FIG. 7 shows a molecular model of thiomethylferrocene (represented as sticks) bonded to a small cluster of gold atoms (spheres) via the thiol group;

FIG. 8 shows a voltage scan as used in linear sweep voltammetry (LSV);

FIG. 9 shows an LSV voltammogram;

FIG. 10 shows a series of linear sweep voltammograms recorded at different scan rates;

FIG. 11 shows a series of voltammograms recorded at a single voltage sweep rate for different values of the reduction rate constant;

FIG. 12 shows a voltage scan as used in cyclic voltammetry (CV);

FIG. 13 shows a typical cyclic voltammogram for a reversible single electrode transfer reaction;

FIG. 14 shows the influence of the voltage scan rate on the current for a reversible electron transfer;

FIG. 15 shows the voltammogram for a quasi-reversible reaction for different values of the reduction and oxidation rate constants;

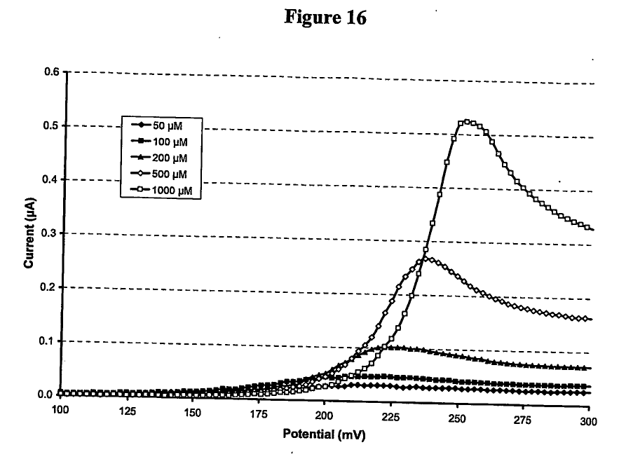

FIG. 16 shows the electrochemical response for an increasing concentration of an example electrochemical mediator (dimethylaminomethylferrocene);

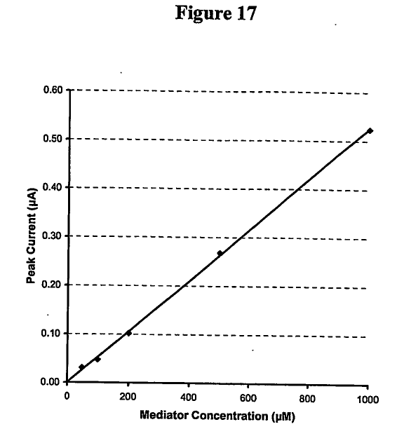

FIG. 17 shows a plot of the maximum peak height in FIG. 16 against mediator concentration;

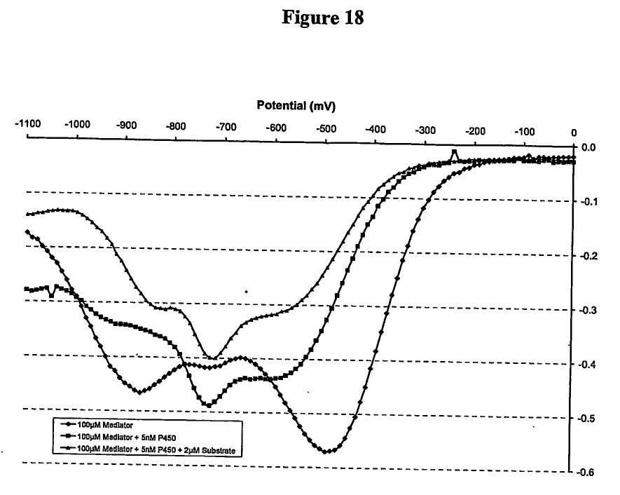

FIG. 18 shows the electrochemical response of FAD mediator; and

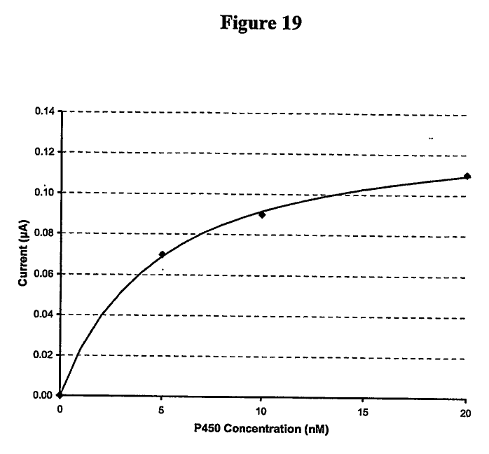

FIG. 19 shows the change in current as P450 concentration is increased.

An embodiment of the invention in which the mediator molecules are in solution is shown diagrammatically in FIG. 3. The mediator molecules shuttle between the electrode surface and the proteins in bulk solution, carrying one or more electrons. Proteins are large molecules, and so diffuse only slowly onto and away from the electrode surface (the ‘mass transport problem’). Being physically much smaller than proteins, the mediators are able to diffuse much more quickly, and therefore the rate of mediated electron transfer is much greater than could be achieved by the proteins themselves.

An alternative embodiment of the invention in which the mediator molecules are immobilised to the electrode to form a protective layer on the surface of the electrode is shown diagrammatically in FIG. 4. Denaturation of the DME molecules caused by direct contact with the surface of the electrode is thereby reduced. The mediator molecules may be covalently or non-covalently immobilised to the electrode.

A particularly preferred embodiment of the invention is shown diagrammatically in FIG. 5. According to this embodiment, the mediator molecules are able to perform two functions: they provide an efficient means to carry electrons from an electrode to DNM molecules in bulk solution, and they protect the protein molecules from denaturation on the electrode surface by forming a protective, electrically-conductive coating layer.

The mediator molecules are designed to have redox and physico-chemical characteristics which are optimised to provide an efficient transfer of electrons for a specific DME, and also a binding site to enable them to form a protective monolayer on the surface of the electrode. Where the binding site forms a non-covalent bond with the electrode, the mediator molecules bound to the electrode will have the same chemical structure as the mediator molecules in solution. Where the binding site forms a covalent bond with the electrode, the mediator bound to the electrode will be the product of covalent binding of the mediator molecules to the electrode. The mediator molecules in solution will be capable of reacting with the electrode to form a covalent bond.

The following examples demonstrate that:

i) we can detect electron transport mediator molecules using electrochemical techniques;

ii) we can accurately quantify the reduction and oxidation of mediators;

iii) we can detect differences in the mediators' electrochemical responses which are induced by the electrochemical driving of cytochrome P450-catalysed reactions; and

iv) the reactions are indeed being driven by the electrochemical mediators.

In the examples below, electrochemical reactions were performed in 100 mM phosphate-buffered saline, at a pH of 7.2. Unless stated otherwise, the proteins were present at 5 nM concentration, and the mediator at 100 μM. The results described in Example 1 are for cytochrome c mediated by dimethylaminomethyl ferrocene, and so are not within the scope of the invention, but illustrate use of an example mediator. The results presented in Examples 2 and 3 are for cytochrome P450 3A4 mediated by flavin adenine dinucleotide (FAD), a flavin-based compound.

All experiments were performed using chemically untreated gold microelectrodes, with a surface area of 0.1 mm² with a reaction chamber volume of 1000 μl. The electrochemical measurement technique employed was cyclic voltammetry, with the potential at the working electrode scanned across the relevant voltage range at a rate of approximately 100 mV per second. A typical scan therefore took around 10 sec to complete, depending on the actual voltage range covered.

### EXAMPLE 1

### Example Mediator Electrochemical Response

The electrochemical response for an increasing concentration of an example electrochemical mediator (dimethylaminomethylferrocene) is shown in FIG. 16. The data was collected using untreated gold electrodes at a pH of 7.2 in phosphate-buffered saline. The results show that there is a clear change in the intensity and position of the peak due to the induced change in the redox state of the mediator. FIG. 17 shows a plot of the maximum peak height against mediator concentration. The plot shows a clear linear response which passes through the origin. This demonstrates that the measured current is directly proportional to the amount of mediator being oxidised (or reduced) by the electrodes. Thus, increasing amounts of mediator generate an increasing electrochemical signal.

### EXAMPLE 2

### Mediated Cytochrome P450: Electrochemically-Driven Reaction

FIG. 18 shows that the electrochemical response of FAD mediator in PBS buffer is changed by the presence of cytochrome P450 (in this case, the 3A4 isozyme), and is further changed by the addition of an appropriate substrate (Vivid® 3A4 Green Fluorogenic Substrate, bivitrogen Ltd.).

The mediator alone gives a voltammogram with two main redox peaks, as would be expected for a flavin-containing compound. Adding the P450 enzyme reduces the magnitude of these peaks, showing that the enzyme is itself interacting with the mediator, and therefore changing the electrochemical response detected at the electrodes. Adding a suitable substrate compound to the mix further reduces the magnitude of the mediator's redox peaks, indicating that the enzyme is now changing the redox state of the mediator even further. This supports the assertion that the mediator is driving the enzyme-catalysed reaction. As was the case in the experiment described in Example 1, the difference in peak magnitudes can be used to monitor the reaction rate. Since each substrate molecule that is oxidised requires the transfer of two electrons, the current difference can be used to calculate the number of reactions catalysed per second (as one Amp is equivalent to 1 mole of electrons transferred each second).

### EXAMPLE 3

### Mediated Cytochrome P450

FIG. 19 shows that at low concentrations, the electrochemical current (indicating the reaction rate) is roughly proportional to enzyme concentration. This is the expected result, and indicates that the overall reaction rate (reactions per second occurring in the reaction chamber) is determined by the amount of enzyme present; the rate-limiting step is the enzyme reaction and not the delivery of electrons. At higher enzyme concentrations, however, the reaction rate tends to a maximum. No matter how much additional enzyme is present, the reaction cannot proceed any faster. This suggests that the rate-limiting step has now become the rate at which electrons can be delivered to the enzyme, and supports the assertion that the mediator is acting in the desired manner (i.e. that the reaction is being driven by the mediator). Were this not the case, the reaction rate would still increase as further enzyme is added.

1. A method of using an electrochemical mediator to transfer electrons from an electrode to molecules of a mammalian oxidative drug metabolizing enzyme (DME) in solution in the absence of a reductase enzyme for the DME molecules, wherein the mediator is in solution with the DME molecules.

2. A method according to claim 1, wherein the electrode is an unmodified electrode.

3. A method according to claim 1, wherein the electrode is coated with a substance through which molecules of the mediator, but not the DME molecules, can diffuse.

4. A method according to claim 1, wherein the mediator comprises a first mediator in solution with the DME molecules, and a second mediator immobilised to the electrode optionally by means of a linker.

5. A method according to claim 4, wherein the second mediator and/or the linker forms a protective layer on the electrode thereby reducing or preventing denaturation of the DME molecules by direct contact with the electrode.

6. A method according to claim 4, wherein the second mediator is non-covalently immobilized to the electrode.

7. A method according to claim 4, wherein the electrode is coated with a substance through which the DME molecules cannot diffuse, and the second mediator is bound to the substance or trapped within it, thereby immobilizing the second mediator to the electrode.

8. A method according to claim 3, wherein the substance is a gel, preferably a polysaccharide gel.

9. A method according to claim 4, wherein the first and second mediators have the same chemical structure.

10. A method according to claim 4, wherein the second mediator is covalently immobilized to the electrode or the linker.

11. A method according to claim 4, wherein the first mediator comprises a functional group which is capable of reacting with the electrode or linker to form a covalent bond with the electrode or linker, and the second mediator is the product of covalent binding of a chemical with the same structure as the first mediator to the electrode or linker.

12. A method according to claim 1, wherein the electrode is a metal electrode.

13. A method according to claim 4, wherein the electrode is a gold electrode, and the immobilized mediator comprises a sulphur-containing group, a pyridine group, a nitrogen-containing heterocyclic group, a carboxylic acid, or a negatively charged moiety by which the mediator is immobilized to the electrode.

14. A method according to claim 1, wherein the DME is a human DME, or a derivative thereof that retains oxidative drug metabolizing activity.

15. A method according to claim 1, wherein the DME is a cytochrome P450 (Cyp), or a derivative thereof that retains oxidative drug metabolizing activity.

16. A method according to claim 1, wherein the or each mediator has a redox potential from about −750 mV to about +750 mV relative to a silver/silver chloride electrode under standard conditions.

17. A method according to claim 1, wherein the or each mediator is capable of transferring electrons from the electrode to the DME molecules at a rate of at least 20 electrons per second.

18. A method according to claim 1, wherein the mediator, or the first and/or the second mediator, comprises any of the following functional groups: an hydroxyl group, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, a halo-, nitro-, phosphor or sulphate group.

19. A method according to claim 1, wherein the mediator, or the first and/or the second mediator, comprises a metallocene, a flavin, a quinone, or NADH, or a redox active derivative thereof.

20. A method according to claim 19, wherein the mediator comprises flavin adenine dinucleotide (FAD), or flavin mononucleotide (FMN), or a redox active derivative thereof.

21. A method according to claim 19, wherein the mediator comprises a ferrocene, or a redox active derivative thereof.

22. A method according to claim 21, wherein the ferrocene is a compound of formula (I): 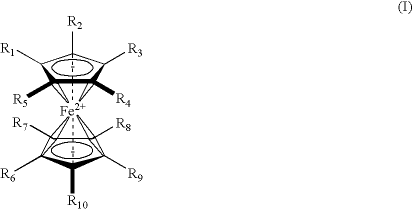
wherein: R₁ is hydrogen; CHO; COCH₃; COCH₂CH₃; COC₃H₆COOH; COCH₂CH₂COOH; CNOHCH₃; COOH; CH₂OH; or CHOHCH₃; and
R₂₋₁₀ are hydrogen.

23. A method according to claim 21, wherein the ferrocene is a compound of formula (II): 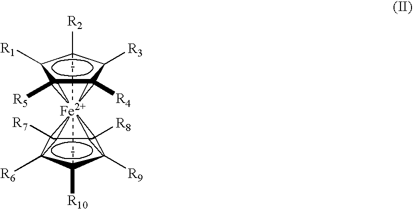
wherein R₁₋₁₀ are independently any of the following: an hydroxyl group, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, a halo-, nitro-, phosphor or sulphate group.

24. A method according to claim 1 in an assay for determining whether a candidate drug is metabolized by the DME.

25. A method according to claim 24, wherein the assay comprises the following steps:
i) providing an electrochemical reaction chamber comprising electrodes, a solution of the DME molecules, the mediator or mediators, and the candidate drug;
ii) applying changing voltage to the electrochemical reaction chamber;
iii) measuring current flowing through the electrochemical reaction chamber; and
iv) determining from the measured current whether the candidate drug is metabolized by the DME.

26. An electrode comprising an electrochemical mediator capable of transferring electrons from the electrode to molecules of a Class II mammalian oxidative DME in solution at a rate which is at least as fast as the reate of consumption of electrons by the molecules when a candidate drug is metabolized by the molecules, wherein the mediator is immobilized to the electrode, optionally by means of a linker, and the mediator and/or the linker forms a protective layer on the electrode thereby reducing or preventing denaturation of the DME molecules caused by direct contact of the DME molecules with the electrode.

27. An electrode comprising an electrochemical mediator capable of transferring electrons from the electrode to molecules of a mammalian oxidative DME in solution, wherein the electrode is coated with a substance through which the DME molecules cannot diffuse, and the mediator is bound to the substance or trapped within it, thereby immobilizing the mediator to the electrode.

28. An electrode according to claim 27, wherein the substance is a gel, preferably a polysaccharide gel.

29. An electrode according to claim 26, wherein the electrode is a metal electrode.

30. An electrode according to claim 26, wherein the immobilized mediator comprises any of the following functional groups: an hydroxyl group, an amide, an amine, a carboxylic acid group, an aromatic group, a cyclic group, a heterocyclic group such as a thiophene, or a nitrogen-containing heterocyclic group such as a pyridine, a purine, or a pyrimidine, an enol, an ether, a ketone, an aldehyde, a thiol, a thioether, a halo-, nitro-, phosphor or sulphate group.

31. An electrode according to claim 26, wherein the immobilized mediator comprises a metallocene, a flavin, a quinone, or NADH, or a redox active derivative thereof.

32. An electrode according to claim 31, wherein the immobilized mediator comprises flavin adenine dinucleotide (FAD), or flavin mononucleotide (FMN), or a redox active derivative thereof.

33. An electrode according to claim 31, wherein the immobilized mediator comprises a ferrocene, or a redox active derivative thereof.

34. An electrode according to claim 26, wherein the immobilized mediator is covalently immobilised to the electrode.

35. An electrode according to claim 34, wherein the electrode is a gold electrode, and the immobized mediator comprises a sulphur-containing group which is covalently attached to the gold.

36. An electrode according to claim 35, wherein the immobilized mediator is the product of covalent binding of thiomethylferrocene to gold.

37. An electrode according to claim 26, wherein the electrode is a gold electrode, and the immobilized mediator comprises a pyridine group, a nitrogen-containing heterocyclic group, a carboxylic acid, or a negatively charged moiety by which the mediator is immobilized to the electrode.

38. An electrode according to claim 26, wherein the immobilized mediator has a redox potential from about −750 mV to about +750 mV relative to a silver/silver chloride electrode under standard conditions.

39. An electrode according to claim 26, wherein the immobilized mediator is capable of transferring electrons from the electrode to the DME molecules at a rate of at least 20 electrons per second.

40. An electrode according to claim 26, wherein the DME is a human DME, or a derivative thereof that retains oxidative drug metabolizing activity.

41. An electrode according to claim 26, wherein the DME is a cytochrome P450 (Cyp), or a derivative thereof that retains oxidative drug metabolizing activity.

42. An electrochemical reaction chamber comprising at least two electrodes, an electrochemical mediator, and molecules of a mammalian DME in solution in the absence of a reductase enzyme for the DME molecules, wherein the mediator is in solution with the DME molecules.

43. A reaction chamber according to claim 42, wherein the mediator comprises a first mediator in solution, and a second mediator immobilized to one or both of the electrodes optionally by means of a linker.

44. A reaction chamber according to claim 43, wherein the second mediator and/or the linker forms a protective layer on the electrode or electrodes thereby reducing or preventing denaturation of the DME molecules caused by direct contact of the DME molecules with the electrode or electrodes.

45. A reaction chamber according to claim 42, wherein one or both of the electrodes are coated with a substance through which molecules of the mediator, but not the DME molecules, can diffuse.

46. A reaction chamber according to claim 43, wherein the electrode or electrodes are coated with a substance through which the DME molecules cannot diffuse, and the second mediator is bound to the substance or trapped within it, thereby immobilizing the second mediator to the electrode.

47. A device comprising a plurality of electrochemical reaction chambers according to claim 42, wherein each electrochemical reaction chamber comprises molecules of a different DME.

48. An assay for identifying an electrochemical mediator for use according to claim 1, which comprises:
i) providing an electrochemical reaction chamber comprising electrodes, a solution of mammalian oxidative DME molecules in the absence of a reductase enzyme for the DME molecules, a substrate for the DME molecules, and a candidate electrochemical mediator in the solution and/or immobilized to one or both of the electrodes;
ii) applying changing voltage to the electrochemical reaction chamber;
iii) measuring current flowing through the electrochemical reaction chamber; and
iv) determining from the measured current the rate of reaction of the substrate with the DME; and
v) comparing the determined rate of reaction with a known rate of reaction of the substrate with the DME under corresponding conditions.

49. An assay according to claim 48, wherein the candidate electrochemical mediator is identified as an electrochemical mediator.
